(19) United States
(12) Patent Application Publication
(10) Pub. No.: US 2014/0154297 A1
(43) Pub. Date: Jun. 5, 2014

(54) ANTIMICROBIAL SUBSTRATES AND METHODS FOR PROCESSING THE SAME

(71) Applicants: Baxter International Inc., (US); Baxter Healthcare S.A., (US)

(72) Inventors: Vadim V. Krongauz, Bartlett, IL (US); Michael Tung-Kiung Ling, Vernon Hills, IL (US); Dustin C. Cawthon, Crystal Lake, IL (US)

(73) Assignees: Baxter Healthcare S.A., Glattpark (Opfikon) (CH); Baxter International Inc., Deerfield, IL (US)

(21) Appl. No.: 13/663,669

(22) Filed: Oct. 30, 2012

(51) Int. Cl. A61K 33/20; A61K 33/18

(52) U.S. Cl. CPC A61K 33/20; A61K 33/18
USPC 424/411; 424/405; 424/661; 424/669

(57) ABSTRACT

Substrates having antimicrobial films or coatings adhered to a surface of the substrate and methods for replenishing the antimicrobial activity of films or coatings adhered to substrate surfaces are disclosed. The antimicrobial films and coatings comprise a strongly basic anion-exchange resin and an anion bound to the strongly basic anion-exchange resin, the anion being free of transition metals. The methods involve exposing a film or coating to an anion having antimicrobial activity, the anion being free of transition metals, and the film or coating comprising a strongly basic anion-exchange resin, thereby obtaining an antimicrobial film or coating comprising the anion bound to the strongly basic anion-exchange resin.

### BACKGROUND

1. Field of the Disclosure

The disclosure relates generally to antimicrobial films and coatings and to methods for processing such films and coatings. More particularly, the disclosure is directed to antimicrobial films and coatings carried on or adhered to a substrate surface, such as a surface of a medical device, and to methods for processing antimicrobial films and coatings to replenish the antimicrobial activity of the film or coating.

2. Brief Description of Related Technology

Even brief exposure to surfaces contaminated with microbes can introduce bacterial, viral, fungal, or other undesirable infections to humans and other animals. Of particular concern is preventing or reducing microbial infection associated with the use of invasive medical devices such as catheters, intravenous fluid administration systems, and other medical devices which require prolonged patient contact and thus present significant infection risks. Contamination may result from the patients' own flora or from one or more healthcare workers' hands during insertion and/or manipulation of the device, or from both the patient and the healthcare worker.

Also of concern are non-invasive devices that may not even directly contact the patient, but which may serve as a vector for transmission of infectious organisms to the patient. Of particular concern are devices that are frequently handled or manipulated by the nurse or caregiver during the course of patient therapy. One example is an intravenous infusion pump, which requires input from the caregiver by pressing buttons or a touch-screen by finger contact. Another example is a stethoscope which is handled by the caregiver and directly contacts the patient. An additional example is a glove, which is also handled by the caregiver and directly contacts the patient. These and like devices can become contaminated with bodily fluids and infection-causing organisms, which may then be transferred directly to the patient or invasive devices used on the patient.

One strategy for reducing contamination is to use antiseptic or disinfecting agents applied directly to the surfaces of medical devices prior to and/or during use. In addition, antiseptics may also be applied to any human tissue that may come into contact with the medical device, particularly percutaneous devices, in an effort to prevent the transfer of infectious organisms to device surfaces.

There are several drawbacks to the use of applied antiseptics and disinfecting agents for preventing contamination of medical devices. One significant drawback relates to the short persistence of antimicrobial activity that is typical of most antiseptics and disinfectants in use in the medical field. Typically, biocidal activity lasts only seconds or minutes, e.g., the time it takes for the agent to dry. This necessitates frequent reapplication of the agent to maintain low contamination on surfaces. For devices that require frequent access, such as needleless connectors, the antiseptic must be applied prior to each access to maintain an effective aseptic barrier. The constant care and attention required to properly maintain these devices puts stress on the resources of hospitals and care facilities, and as a result, antisepsis is often neglected.

Another drawback to the use of antiseptic and disinfecting agents to control contamination on medical devices is the lack of control over their effectiveness, which is dependent upon application technique and specific device characteristics. Factors such as exposure time, application pressure, drying time, surface topography, material properties, and the level of existing surface contamination can have a significant impact on biocidal activity. Given the aforementioned drawbacks to using applied antiseptic and disinfecting agents for antisepsis, some manufacturers have responded by applying antimicrobial coatings to the surfaces of medical devices. The coatings are designed to provide extended antimicrobial protection that is not dependent upon the caregiver having to remember to disinfect the surface or to use a specific technique. They are also not affected by device surface or material characteristics because these parameters are factored in during design.

Medical devices coated with antimicrobial materials can reduce the transfer of microbes to patients, thereby improving the safety and efficacy of these devices. Such antimicrobial coatings often include silver metal or silver salts, or other metals with demonstrable antimicrobial activity such as copper, gold, zinc, cerium, platinum, palladium, or tin. Antimicrobial coatings comprising metals and metal salts, such as silver metal and silver salts, however, frequently do not provide activity sufficiently fast enough and/or for a sufficiently long enough period of time to effectively and efficiently control microbial contamination. This is especially true for devices that are frequently handled or accessed, and devices that are used for extended periods of time (e.g., weeks, months, or years). Therefore, there is a need for a material with antiseptic properties having biocidal efficacy rapid enough to sufficiently control contamination on touch surfaces or on surfaces that come into contact with body fluids. There is also a need for a material with antiseptic properties having sufficient persistence of biocidal efficacy to be used on devices for extended periods of time. Preferably, the material would possess the ability for the antimicrobial agent to be replenished. This would facilitate use on devices subjected to demanding use conditions, such as those subjected to heavy contamination by debris, biological fluids, or microbial contamination; devices that are handled or accessed frequently; or durable medical devices, such as infusion pumps, that may be used for years.

### SUMMARY

In one aspect, the present disclosure is directed to a substrate having a film or coating adhered to a surface of the substrate, the film or coating comprising a strongly basic anion-exchange resin comprising quaternary ammonium functional groups and an anion bound to the strongly basic anion-exchange resin, the anion being free of transition metals, and the film or coating having antimicrobial activity. In another aspect, the present disclosure is directed to a method of replenishing the antimicrobial activity of a film or coating comprising exposing a film or coating adhered to a substrate surface to an anion having antimicrobial activity, the anion being free of transition metals, and the film or coating comprising a strongly basic anion-exchange resin, thereby obtaining an antimicrobial film or coating comprising the anion bound to the strongly basic anion-exchange resin.

The substrate surfaces can comprise plastic, glass, metal, ceramics, elastomers, or mixtures or laminates thereof. The substrate surfaces can comprise surfaces of medical devices or medical device components. Preferred examples of substrate surfaces include polycarbonate medical devices, polypropylene medical devices, silicone medical devices, polyurethane medical devices, polyester medical devices, and polyvinyl chloride medical devices. The substrate surface also can comprise surfaces of invasive medical devices, durable medical devices, medical fluid containers, or medical fluid flow systems. Examples of durable medical devices include intravenous (I.V.) pumps, patient monitors, stethoscopes, and I.V. poles. Examples of medical fluid flow systems include I.V. sets, intraperitoneal sets, and components thereof, such as, for example, luer access devices.

In various embodiments, the anion has antimicrobial activity. Preferably, the anion is free of metals. Suitable anions include, but are not limited to, anions comprising an inorganic anion or a halogen atom. For example, the anion can be selected from the group consisting of I⁻, IO₃⁻, Cl⁻, ClO⁻, ClO₃⁻, Br⁻, BrO₃⁻, F⁻, and mixtures thereof.

In various embodiments, the film or coating comprising the strongly basic anion-exchange resin has a thickness of about 25 μm to about 10 mm.

In various embodiments, the exposing step comprises applying bleach, Lugol's iodine, or povidone-iodine to the film or coating.

### DETAILED DESCRIPTION

The present disclosure advantageously provides a substrate having a fast-acting antimicrobial film or coating adhered to a surface of the substrate, the film or coating comprising a strongly basic anion-exchange resin and an anion bound to the strongly basic anion-exchange resin. Moreover, the disclosed antimicrobial films and coatings are beneficially capable of having their antimicrobial activity replenished quickly and easily.

The disclosed films and coatings are adhered to a substrate surface and in each instance the films and coatings have antimicrobial activity. As used herein, the term “adhered to” means in direct contact with, present on, and/or carried by a substrate surface. The films and coatings according to the disclosure comprise a strongly basic anion-exchange resin and an anion bound to the strongly basic anion-exchange resin, the anion being free of transition metals.

The present disclosure also is directed to methods for replenishing the antimicrobial activity of such films and coatings. The methods comprise exposing a film or coating adhered to a substrate surface to an anion having antimicrobial activity, the anion being free of transition metals, and the film or coating comprising a strongly basic anion-exchange resin, thereby obtaining an antimicrobial film or coating comprising the anion bound to the strongly basic anion-exchange resin.

Suitable anion-exchange resins include resins comprising repeating functional groups capable of reversibly binding to various anions. As is well known, typical anion-exchange resins consist of multiple functional groups bound to an insoluble matrix comprising an organic polymer. Frequently used polymers for anion-exchange resins include, but are not limited to, polystyrene. Anion-exchange resins generally are manufactured in the form of small beads (e.g., 0.05 mm to 5 mm in diameter), thin sheets, films, and capillary tubing. The terms strong anion-exchange resin and strongly basic anion-exchange resin are used interchangeably herein. Strong anion-exchange resins include resins comprising repeating quaternary ammonium functional groups. Commercially available strong anion-exchange resins include, but are not limited to, trialkylbenzyl ammonium anion-exchange resins such as AMBERLITE Type I anion-exchange resin (Rohm and Haas Co.), trimethylbenzyl ammonium anion-exchange resins such as DOWEX Type I anion-exchange resin (Dow Chemical Co.), and dimethyl-2-hydroxyethylbenzyl ammonium anion-exchange resins such as AMBERLITE Type II anion-exchange resin (Rohm and Haas Co.) and DOWEX Type II anion-exchange resin (Dow Chemical Co.).

In some embodiments the anion-exchange resin has demonstrable antimicrobial activity. In some embodiments an anion-exchange resin having demonstrable antimicrobial activity is used in combination with one or more anions having demonstrable antimicrobial activity. Typically, at least the anion has demonstrable antimicrobial activity. “Demonstrable antimicrobial activity” as used herein typically refers to at least a 10-fold (e.g., at least a 50-fold and/or at least a 100-fold) reduction in microbial levels. An exemplary method for assessing reduction in microbial levels is described in the examples herein.

Suitable anions are free of transition metals (e.g., the anions are free of elemental transition metals such as anionic complexes of elemental transition metals and/or the anions are free of transition metal ions such as anionic complexes of transition metal ions). While various transition metal ions (e.g., silver ions) demonstrate antimicrobial activity, transition metals demonstrate several disadvantages when used as components of antimicrobial coatings. One disadvantage of some transition metal-containing antimicrobial coatings is their color/opaqueness, which prevents a healthcare provider from being able to see through the medical device substrate. Coatings comprising metallic silver, for example, can be brown in color. Thus, when such colored coatings are applied to transparent surfaces, the coated surfaces typically have a brown color and significantly diminished transparency. Another disadvantage of some metal-containing antimicrobial coatings is the relatively short duration of effective antimicrobial activity demonstrated by the coatings and the inability to easily replenish the activity of such coatings. For example, the antimicrobial activity of silver-containing antimicrobial coatings can be depleted over a few days to a week. Thus, when long-term antimicrobial activity is desired, such silver-containing antimicrobial coatings typically would not provide effective antimicrobial activity for the entire desired time period.

In some embodiments, the anions are free of metals (e.g., elemental metals and metal ions). For example, the anions are free of transition metals, alkali metals, alkaline earth metals, and other metals including, but not limited to, aluminum and tin. In some embodiments the anions consist exclusively of non-metals, halogens, and mixtures thereof.

The anions can be selected from inorganic anions, organic anions, and mixtures thereof. The anions typically include anions comprising one or more halogens, such as F, Cl, Br, and I. Exemplary anions include, but are not limited to, I⁻, IO₃⁻, Cl⁻, ClO⁻, ClO₃⁻, Br⁻, BrO₃⁻, F⁻, and mixtures thereof. In some embodiments, the films or coatings include a combination of two or more anions.

### Substrate Surfaces

The substrate surfaces of the present disclosure can comprise various materials including, for example, glasses, metals, plastics, ceramics, and elastomers, as well as mixtures and/or laminates thereof. Suitable examples of plastics include, but are not limited to, acrylonitrile butadiene styrenes, polyacrylonitriles, polyamides, polycarbonates, polyesters, polyetheretherketones, polyetherimides, polyethylenes such as high density polyethylenes and low density polyethylenes, polyethylene terephthalates, polylactic acids, polymethyl methyacrylates, polypropylenes, polystyrenes, polyurethanes, poly(vinyl chlorides), polyvinylidene chlorides, polyethers, polysulfones, silicones, and blends and copolymers thereof. Suitable elastomers include, but are not limited to, natural rubbers and synthetic rubbers, such as styrene butadiene rubbers, ethylene propylene diene monomer rubbers (EPDM), polychloroprene rubbers (CR), acrylonitrile butadiene rubbers (NBR), chlorosulphonated polyethylene rubbers (CSM), polyisoprene rubbers, isobutylene-isoprene copolymeric rubbers, chlorinated isobutylene-isoprene copolymeric rubbers, brominated isobutylene-isoprene copolymeric rubbers, and blends and copolymers thereof.

In one preferred embodiment of the present disclosure, the film or coating comprising a strongly basic anion-exchange resin and an anion bound to the strongly basic anion-exchange resin is in direct contact with, present on, carried by, and/or adhered to a surface of a medical device or medical device component. Medical devices and medical device components which can benefit from the methods according to the disclosure, include, but are not limited to, instruments, apparatuses, implements, machines, contrivances, implants, and components and accessories thereof, intended for use in the diagnosis, cure, mitigation, treatment, or prevention of disease or other condition in humans or other animals, or intended to affect the structure or any function of the body of humans or other animals. Such medical devices are described, for example, in the official National Formulary, the United States Pharmacopoeia, and any supplements thereto. Suitable medical devices include devices that are handled or accessed routinely during the course of patient care (and thus may serve as a point of microbe transfer from device to device or from device to patient), and include devices that require frequent cleansing with antiseptic or disinfecting agents, including, but not limited to percutaneous devices and durable medical devices.

Representative medical devices include, but are not limited to: catheters, such as venous catheters, urinary catheters, Foley catheters, and pain management catheters; dialysis sets; dialysis connectors; stents; abdominal plugs; feeding tubes; indwelling devices; cotton gauzes; wound dressings; contact lenses; lens cases; bandages; sutures; hernia meshes; mesh-based wound coverings; surgical tools; medical monitoring equipment including, but not limited to the touch screen displays often used in conjunction with such equipment; medical pumps; pump housings; gaskets such as silicone O-rings; needles; syringes; surgical sutures; filtration devices; drug reconstitution devices; implants; metal screws; and metal plates.

Additional exemplary medical devices include, but are not limited to, invasive medical devices, durable medical devices, medical fluid containers, medical fluid flow systems, infusion pumps, patient monitors, and medical devices such as stethoscopes which regularly come into contact with a patient. Examples of medical fluid flow systems are an intravenous fluid administration set, also known as an I.V. set, used for the intravenous administration of fluids to a patient, and an intraperitoneal administration set, also known as an intraperitoneal set, used for the intraperitoneal administration of fluids to a patient. A typical I.V. set uses plastic tubing to connect a phlebotomized subject to one or more medical fluid sources, such as intravenous solutions or medicament containers. I.V. sets optionally include one or more access devices providing access to the fluid flow path to allow fluid to be added to or withdrawn from the IV tubing. Access devices advantageously eliminate the need to repeatedly phlebotomize the subject and allow for immediate administration of medication or other fluids to the subject, as is well known. Access devices can be designed for use with connecting apparatus employing standard luers, and such devices are commonly referred to as “luer access devices,” “luer-activated devices,” or “LADs.” LADs can be modified with one or more features such as antiseptic indicating devices. Various LADs are illustrated in U.S. Pat. Nos. 5,242,432, 5,360,413, 5,730,418, 5,782,816, 6,039,302, 6,669,681, and 6,682,509, and U.S. Patent Application Publication Nos. 2003/0141477, 2003/0208165, 2008/0021381, and 2008/0021392, the disclosures of which are hereby incorporated by reference in their entireties.

I.V. sets or intraperitoneal sets can incorporate additional optional components including, for example, septa, stoppers, stopcocks, connectors, protective connector caps, connector closures, adaptors, clamps, extension sets, filters, and the like. Thus, additional suitable medical devices and medical device components which may be processed in accordance with the methods of the present disclosure include, but are not limited to: I.V. tubing, I.V. fluid bags, I.V. set access devices, septa, stopcocks, I.V. set connectors, I.V. set connector caps, I.V. set connector closures, I.V. set adaptors, clamps, I.V. filters, I.V. pumps, I.V. poles, catheters, needles, cannulae, stethoscopes, patient monitors, intraperitoneal tubing, intraperitoneal fluid bags, access devices for intraperitoneal sets, intraperitoneal set connectors, intraperitoneal set adaptors, and intraperitoneal filters. Representative access devices include, but are not limited to: luer access devices including, but not limited to, needleless luer access devices.

Additional exemplary medical devices include, but are not limited to: intraurethral catheters, intra-arterial catheters, intraosseous catheters, intrathecal catheters, intra-pulmonary catheters, tracheal tubes, nasogastric tubes, and components thereof.

The surface of the medical device or medical device component can be fully or partially covered with the film or coating comprising a strongly basic anion-exchange resin and an anion bound to the strongly basic anion-exchange resin. The film or coating can be in direct contact with, present on, carried by, and/or adhered to an exterior surface of the device (i.e., a surface which is intended to come into contact with a patient or healthcare provider), an interior surface of the device (i.e., a surface which is not intended to come into contact with a patient or healthcare provider, but which can come into contact with the patient's blood or other fluids), or both. Suitable medical devices and medical device components are illustrated in U.S. Pat. Nos. 4,412,834, 4,417,890, 4,440,207, 4,457,749, 4,485,064, 4,592,920, 4,603,152, 4,738,668, 5,630,804, 5,928,174, 5,948,385, 6,355,858, 6,592,814, 6,605,751, 6,780,332, 6,800,278, 6,849,214, 6,878,757, 6,897,349, 6,921,390, and 6,984,392, and U.S. Patent Application Publication No. 2007/0085036, the disclosures of which are hereby incorporated by reference in their entireties.

The substrate surfaces carrying films or coatings comprising an anion-exchange resin and an anion bound to the anion-exchange resin can be produced by a wide variety of known methods for adhering films and coatings to surfaces. Known techniques for producing such films and coatings include, for example, wiping, brushing, spraying, and dipping. Methods for adhering films and coatings to substrate surfaces include providing a solution or suspension of the film or coating components in a solvent, exposing a substrate surface to the solution or suspension (e.g., by wiping, brushing, spraying, or dipping), and removing the solvent. Methods for removing the solvent include, for example, allowing the solvent to evaporate with or without heating the substrate surface to evaporate the solvent. Methods for adhering films and coatings to substrate surfaces also include exposing a substrate surface to a mixture consisting exclusively of the film or coating components (e.g., by wiping, brushing, spraying, or dipping) in the absence of a solvent.

The films and coatings adhered to the substrate surfaces include films and coatings having various thicknesses. The films and coatings adhered to the substrate surfaces include films and coatings having a thickness of about 25 μm to about 10 mm, about 25 μm to about 5 mm, about 25 μm to about 1 mm and/or about 25 μm to about 100 μm.

### Methods For Replenishing the Antimicrobial Activity of the Films and Coatings

As discussed previously, some antimicrobial coatings such as silver-containing antimicrobial coatings can demonstrate antimicrobial activity for an insufficient period of time. While not intending to be bound by theory, decreased antimicrobial activity of such silver-containing coatings often results from depletion of a coating component (e.g., silver ions) having antimicrobial activity. The present disclosure addresses this problem by providing films and coatings capable of having their antimicrobial activity replenished and by providing methods for processing such films and coatings to replenish the antimicrobial activity of the films and coatings.

The films and coatings capable of having their antimicrobial activity replenished comprise a strongly basic anion-exchange resin and are adhered to a substrate surface. Processing these films and coatings according to the disclosed methods replenishes the antimicrobial activity of the films and coatings, thereby significantly extending the duration of antimicrobial activity demonstrated by the film or coating, particularly as compared to the duration of antimicrobial activity demonstrated by the same film or coating which has not been processed according to the disclosed methods.

In some embodiments, the antimicrobial activity of the films or coatings is replenished according to the disclosed methods when the antimicrobial activity of the film or coating has diminished to an undesirably low level. Undesirably low antimicrobial activity includes antimicrobial activity that is less than 90% of the initial antimicrobial activity, for example, less than 80%, less than 70%, less than 60%, less than 50%, less than 40%, less than 30%, less than 20%, less than 10%, and/or less than 5% of the initial antimicrobial activity. Suitable methods for measuring the antimicrobial activity of the films and coatings include a variety of known methods for measuring antimicrobial activity. In some embodiments, the antimicrobial activity of the film or coating is replenished according to the disclosed methods before the antimicrobial activity of the film or coating has diminished significantly. For example, the film or coating can be processed before the antimicrobial activity of the film or coating has diminished to less than 90% of the initial antimicrobial activity. Processing the film or coating according to the disclosed methods when the antimicrobial activity of the film or coating is still at a high level, such as a level that is 90% or more of the initial antimicrobial activity, maintains the film's or coating's high level of antimicrobial activity. Such prophylactic processing of the films or coatings according to the disclosed methods on a regular basis, for example, on a daily or weekly basis, decreases the risk that the antimicrobial activity of a film or coating will fall to an undesirably low level and avoids the inconvenience and cost associated with frequent testing of the antimicrobial films and coatings to determine their antimicrobial activity. Advantageously, the disclosed methods include processing the films or coatings multiple times (e.g., two, three, four, five, ten, twenty, or more times) throughout the useful lifetime of the substrate surface to which the film or coating is adhered, thereby maintaining the antimicrobial activity of the film or coating for an extended period of time.

The methods for replenishing the antimicrobial activity of the films and coatings adhered to a substrate surface include exposing the film or coating to an anion having antimicrobial activity. Methods for exposing films and coatings to an anion having antimicrobial activity include, for example, contacting the substrate surface with the anion by wiping, brushing, spraying, and/or dipping. For example, the methods include providing a solution or suspension of the anion in a solvent, exposing a substrate surface to the solution or suspension (e.g., by wiping, brushing, spraying, or dipping), and removing the solvent. Methods for removing the solvent include, for example, allowing the solvent to evaporate with or without heating the substrate surface to evaporate the solvent. Methods for exposing films and coatings to an anion having antimicrobial activity also include contacting a substrate surface with the anion (e.g., by wiping, brushing, spraying, or dipping) in the absence of a solvent.

The anions having antimicrobial activity include both inorganic anions and organic anions. The anions typically include anions comprising one or more halogen atoms, such as the halogen atoms F, Cl, Br, and I. Exemplary anions include, but are not limited to, I⁻, IO₃⁻, Cl⁻, ClO⁻, ClO₃⁻, Br⁻, BrO₃⁻, F⁻, and mixtures thereof. In some embodiments, the exposing step comprises exposing the film or coating to a combination of two or more anions having antimicrobial activity.

In some embodiments, the exposing step comprises exposing the film or coating to a source of anions including, but not limited to, a hypochlorite solution (e.g., sodium hypochlorite or bleach), a chloride solution (e.g., potassium chloride, sodium chloride), an iodide solution (e.g., potassium iodide, sodium iodide), a molecular iodine solution, a solution of molecular iodine and iodide (e.g., Lugol's iodine), or a povidone-iodine solution.

Typically, the substrate surface is exposed to the anion for less than about 5 minutes, but longer exposure periods can be used. Generally, the substrate surface is exposed to the anion for less than about 3 minutes, less than about 1 minute, less than about 45 seconds, less than about 30 seconds, less than about 20 seconds, and/or less than about 15 seconds, more preferably, less than about 10 seconds, less than about 5 seconds, less than about 3 seconds, and/or less than about 1 second.

After processing a substrate surface having a film or coating comprising a strongly basic anion-exchange resin in accordance with the present methods, the antimicrobial activity of the processed film or coating is typically at least 5% greater than the antimicrobial activity of the film or coating before processing the substrate surface in accordance with the present methods. Generally, the antimicrobial activity after processing by exposure to the anion is more than 5% greater than the antimicrobial activity prior to exposure. For example, the antimicrobial activity after exposure can be at least 10%, at least 20%, at least 40%, at least 60%, and/or at least 80% greater than the antimicrobial activity prior to processing. Additionally, after processing a substrate surface having a film or coating comprising an anion-exchange resin by exposing to an anion having antimicrobial activity in accordance with the present methods, the film or coating typically has an increased amount of the anion (bound to the strongly basic anion-exchange resin or otherwise associated with the film or coating), compared to the amount of anion in the film or coating prior to processing by exposure to the anion.

The disclosure may be better understood by reference to the following examples which are not intended to be limiting, but rather only set forth exemplary embodiments in accordance with the disclosure.

### EXAMPLES

### Example 1

### Preparation of Anion-Exchange Resins

To 20 g of DOWEX® 1X4, chloride form, strongly basic anion-exchange resin (Sigma Aldrich) was added 200 g CLOROX COMMERCIAL SOLUTIONS® bleach. The resulting mixture was shaken for 24 hours. The mixture was then filtered using a glass frit filter under vacuum and rinsed four times using a total of 200 mL distilled water, thereby obtaining a bleach-treated anion-exchange resin.

To 20 g of DOWEX® 1X4, chloride form, strongly basic anion-exchange resin (Sigma Aldrich) was added 200 g of povidone iodine solution (10.5% PVP-iodine diluted 1:10 with distilled water) (Baxter International). The resulting mixture was shaken for 24 hours. The mixture was then filtered using a glass frit filter under vacuum and rinsed four times using a total of 200 mL distilled water, thereby obtaining a povidone iodine-treated anion-exchange resin.

As a control, the strongly basic anion-exchange resin was processed with sodium hydroxide to exchange chloride ions with hydroxide ions. Specifically, to 20 g of DOWEX® 1X4, chloride form, strongly basic anion-exchange resin (Sigma Aldrich) was added 200 g 1.002 N sodium hydroxide. The resulting mixture was shaken for 24 hours. The mixture was then filtered using a glass frit filter under vacuum and rinsed four times using a total of 200 mL distilled water, thereby obtaining a sodium hydroxide-treated anion-exchange resin.

### Example 2

### Antimicrobial Activity of Anion-Exchange Resins

The antimicrobial activity of the anion-exchange resins prepared in Example 1 against Staphylococcus aureus ATCC 6538 (S. aureus) was tested. A suspension of S. aureus was grown in soybean-casein digest (SCD) broth for approximately 24 hours. The suspension was then diluted in sterile saline to a working concentration of approximately 10⁵ colony-forming units per mL (CFU/mL). For each exposure point evaluated, triplicate sterile tubes containing 5 mL sterile saline were inoculated with 0.1 mL (approximately 10⁴ CFU) of the working suspension. 0.5 g of treated resin was aseptically added to each of the tubes, which were held at 20-25° C. for 0.25, 6, 24, or 48 hours under dynamic conditions on an orbital shaker set at 250 RPM. As a control, tubes containing only sterile saline (no added resin) were inoculated with the working suspension and held at 20-25° C. for 0.25, 6, 24, or 48 hours in the same manner, and concurrent with, the test articles. The samples then were pour plated in duplicate using SCD agar and incubated at 30-35° C. for a minimum of 48 hours. After this time, the CFU of S. aureus were enumerated, as shown in Tables 1 to 4.

TABLE 1

| | Sample 1 | Sample 2 | Sample 3 | | log |
|---|---|---|---|---|---|
| Sample | Recovery | Recovery | Recovery | Average | (Aver- |
| (0.25 hrs) | (cfu) | (cfu) | (cfu) | (cfu) | age) |
| Saline | 7.3 × 10⁴ | 8.5 × 10⁴ | — | 7.9 × 10⁴ | 4.90 |
| control | | | | | |
| Bleach- | 1.5 × 10¹ | 2.1 × 10¹ | 3.2 × 10¹ | 2.3 × 10¹ | 1.36 |
| treated | | | | | |
| resin | | | | | |
| PVP-iodine- | 2.1 × 10⁴ | 3.1 × 10⁴ | 2.5 × 10⁴ | 2.6 × 10⁴ | 4.41 |
| treated resin | | | | | |

TABLE 2

| | Sample 1 | Sample 2 | Sample 3 | | log |
|---|---|---|---|---|---|
| Sample | Recovery | Recovery | Recovery | Average | (Aver- |
| (6 hrs) | (cfu) | (cfu) | (cfu) | (cfu) | age) |
| Saline | 1.1 × 10⁴ | 7.5 × 10³ | — | 9.3 × 10³ | 3.97 |
| control | | | | | |
| NaOH- | 2.7 × 10⁴ | 3.0 × 10⁴ | 2.2 × 10⁴ | 2.6 × 10⁴ | 4.41 |
| treated | | | | | |
| resin | | | | | |
| Bleach- | 0 | 0 | 1.0 × 10⁰ | 3.3 × 10⁻¹ | −0.48 |
| treated | | | | | |
| resin | | | | | |
| PVP-iodine- | 0 | 0 | 0 | 0 | N/A |
| treated resin | | | | | |

TABLE 3

| | Sample 1 | Sample 2 | Sample 3 | | log |
|---|---|---|---|---|---|
| Sample | Recovery | Recovery | Recovery | Average | (Aver- |
| (24 hrs) | (cfu) | (cfu) | (cfu) | (cfu) | age) |
| Saline | 2.6 × 10³ | 4.5 × 10³ | — | 3.6 × 10³ | 3.56 |
| control | | | | | |
| NaOH- | 1.9 × 10⁴ | 1.7 × 10⁴ | 2.1 × 10⁴ | 1.9 × 10⁴ | 4.28 |
| treated | | | | | |
| resin | | | | | |
| Bleach- | 1.0 × 10⁰ | 0 | 3.0 × 10⁰ | 1.3 × 10⁰ | 0.11 |
| treated | | | | | |
| resin | | | | | |
| PVP-iodine- | 1.0 × 10⁰ | 0 | 2.0 × 10⁰ | 1.1 × 10⁰ | 0.04 |
| treated resin | | | | | |

TABLE 4

| | Sample 1 | Sample 2 | Sample 3 | | log |
|---|---|---|---|---|---|
| Sample | Recovery | Recovery | Recovery | Average | (Aver- |
| (48 hrs) | (cfu) | (cfu) | (cfu) | (cfu) | age) |
| Saline | 2.5 × 10³ | 3.8 × 10³ | — | 3.2 × 10³ | 3.51 |
| control | | | | | |
| NaOH- | 5.2 × 10³ | 1.0 × 10⁴ | 5.3 × 10³ | 6.8 × 10³ | 3.83 |
| treated | | | | | |
| resin | | | | | |
| Bleach- | 0 | 0 | 0 | 0 | N/A |
| treated | | | | | |
| resin | | | | | |
| PVP-iodine- | 5 × 10⁰ | 0 | 0 | 1.7 × 10⁰ | 0.23 |
| treated resin | | | | | |

Table 5 shows the resulting log reduction value for the samples, which is obtained by the following calculation: log reduction value=log A_{control}(t)−log Aₛₐₘₚₗₑ(t), where A_{control}(t) is the average CFU for the saline control at time t and Aₛₐₘₚₗₑ(t) is the average CFU for the treated resin sample at time t.

TABLE 5

| | NaOH-treated | Bleach-treated | PVP-iodine-treated |
|---|---|---|---|
| Time (h) | resin | resin | resin |
| 0.25 | — | 3.54 | 0.49 |
| 6 | −0.45 | ≧3.97 | ≧3.97 |
| 24 | −0.72 | 3.45 | 3.52 |
| 48 | −0.32 | ≧3.51 | 3.28 |

The data in Table 5 demonstrate that when contacted with S. aureus for 6, 24, or 48 hours, both the bleach-treated resin and PVP-iodine-treated resin demonstrated significant antimicrobial activity against S. aureus, as compared to saline-only control samples. Further, the bleach-treated resin demonstrated significant antimicrobial activity against S. aureus, as compared to saline-only control samples, after only 0.25 hours. Additionally, the data in Table 5 demonstrate that the antimicrobial activity of the NaOH-treated resin was similar to the saline-only control samples.

### Example 3

### Recharging the Antimicrobial Activity of Anion-Exchange Resins

In a prophetic example, an antimicrobial film or coating comprising a strongly basic anion-exchange resin and having diminished antimicrobial activity is processed according to the disclosed methods to replenish the antimicrobial activity of the film or coating. Specifically, the antimicrobial activity of the film or coating is replenished by wiping, brushing, or spraying the film or coating with an aqueous solution of a suitable anion or by immersing a substrate surface carrying the antimicrobial film or coating in an aqueous solution of a suitable anion. The aqueous solution is a hypochlorite solution such as a sodium hypochlorite solution (i.e., bleach), a chloride solution such as a chloride salt solution, an iodide solution such as an iodide salt solution, or a povidone-iodine solution.

1. A substrate having a film or coating adhered to a surface of the substrate, the film or coating comprising:
(i) a strongly basic anion-exchange resin comprising quaternary ammonium functional groups and
(ii) an anion bound to the strongly basic anion-exchange resin, the anion being free of transition metals, and the film or coating having antimicrobial activity.

2. The substrate of claim 1, wherein the substrate surface comprises at least one plastic, glass, metal, ceramic, elastomer, or mixtures or laminates thereof.

3. The substrate of claim 1, wherein the substrate surface comprises a plastic or elastomer selected from the group consisting of: acrylonitrile butadiene styrenes, polyacrylonitriles, polyamides, polycarbonates, polyesters, polyetheretherketones, polyetherimides, polyethylenes, polyethylene terephthalates, polylactic acids, polymethyl methyacrylates, polypropylenes, polystyrenes, polyurethanes, poly(vinyl chlorides), polyvinylidene chlorides, polyethers, polysulfones, silicones, natural rubbers, synthetic rubbers, styrene butadiene rubbers, ethylene propylene diene monomer rubbers, polychloroprene rubbers, acrylonitrile butadiene rubbers, chlorosulphonated polyethylene rubbers, polyisoprene rubbers, isobutylene-isoprene copolymeric rubbers, chlorinated isobutylene-isoprene copolymeric rubbers, brominated isobutylene-isoprene copolymeric rubbers, and blends and copolymers thereof.

4. The substrate of claim 1, wherein the substrate surface comprises a surface of a medical device or medical device component.

5. The substrate of claim 1, wherein the substrate surface comprises a surface of an invasive medical device, a durable medical device, a medical fluid container, or medical fluid flow system.

6. The substrate of claim 1, wherein the substrate surface comprises a surface of an I.V. set or an intraperitoneal set.

7. The substrate of claim 1, wherein the substrate surface comprises a surface of a medical device or medical device component selected from the group consisting of: I.V. tubing, I.V. fluid bags, access devices for I.V. sets, septa, stopcocks, I.V. set connectors, I.V. set adaptors, clamps, I.V. filters, I.V. pumps, I.V. poles, catheters, needles, cannulae, stethoscopes, patient monitors, intraperitoneal tubing, intraperitoneal fluid bags, access devices for intraperitoneal sets, intraperitoneal set connectors, intraperitoneal set adaptors, and intraperitoneal filters.

8. The substrate of claim 1, wherein the substrate surface comprises a surface of a medical device or medical device component selected from the group consisting of: intraurethral catheters, intra-arterial catheters, intraosseous catheters, intrathecal catheters, intra-pulmonary catheters, tracheal tubes, nasogastric tubes, and components thereof.

9. The substrate of claim 1, wherein the substrate surface comprises a surface of a luer access device or a needleless luer access device.

10. The substrate of claim 1, wherein the anion has antimicrobial activity.

11. The substrate of claim 1, wherein the anion is free of metals.

12. The substrate of claim 1, wherein the anion comprises an inorganic anion.

13. The substrate of claim 1, wherein the anion comprises a halogen.

14. The substrate of claim 1, wherein the anion is selected from the group consisting of I⁻, IO₃⁻, Cl⁻, ClO⁻, ClO₃⁻, Br⁻, BrO₃⁻, F⁻, and mixtures thereof.

15. The substrate of claim 1, wherein the film or coating has a thickness of about 25 μm to about 10 mm.

16. A method of replenishing the antimicrobial activity of a film or coating comprising: exposing a film or coating adhered to a substrate surface to an anion having antimicrobial activity, the anion being free of transition metals, and the film or coating comprising a strongly basic anion-exchange resin comprising quaternary ammonium functional groups, thereby obtaining an antimicrobial film or coating comprising the anion bound to the strongly basic anion-exchange resin.

17. The method of claim 16, wherein the anion is hypochlorite or iodide.

18. The method of claim 16, wherein the exposing step comprises applying bleach, Lugol's iodine, or povidone-iodine to the film or coating.

19. The method of claim 16, wherein the substrate surface comprises a surface of a medical device or medical device component.

20. The method of claim 16, wherein the film or coating has a thickness of about 25 μm to about 10 mm.
